(19) United States
(12) Patent Application Publication
(10) Pub. No.: US 2025/0169689 A1
(43) Pub. Date: May 29, 2025

(54) ENDOSCOPE WITH SENSOR FOR IMAGE CAPTURING AND TISSUE EXAMINATION

(71) Applicant: Erbe Elektromedizin GmbH, Tuebingen (DE)

(72) Inventors: Jan JAEGER, Freiburg im Breisgau (DE); Reza MOHAMMADI, Reutlingen (DE)

(21) Appl. No.: 18/953,474

(22) Filed: Nov. 20, 2024

(30) Foreign Application Priority Data
Nov. 24, 2023 (EP) 23211973.5
Nov. 5, 2024 (EP) 24210937.9

(51) Int. Cl. A61B 1/04; A61B 1/06

(52) U.S. Cl. CPC A61B 1/046; A61B 1/0676

(57) ABSTRACT

An endoscope that is configured for imaging as well as for tissue analysis is disclosed. The endoscope includes an image sensor to which multiple optical devices are arranged in order to carry out different tasks with one and the same image sensor. In particular, the task of imaging and measurement tasks may be concurrently or temporally graded.

### CROSS REFERENCE TO RELATED APPLICATIONS

This application claims the benefit of European Patent Application No. 23211973.5, filed Nov. 24, 2023, and European Patent Application No. 24210937.9, filed Nov. 5, 2024, both of which are incorporated herein by reference in their entirety.

### TECHNICAL FIELD

The invention refers to an endoscope for medical examination and/or treatment of a human or animal body or tissue sections thereof.

### BACKGROUND

For examination and treatment of human or animal patients endoscopes are known, which comprise a longitudinal shank that can be inserted into the body of the patient.

For example, EP 2 075 617 A1 discloses an endoscope with an optical device for spectral examination of the tissue present in front of the distal end of the endoscope. For this purpose, the endoscope comprises a through-hole at its distal end, serving as light entrance window. By means of piezoelectrical elements, a variable filter can be influenced, so that the wavelength of the passing light can be modified. In addition, an illumination device and an image capturing device arranged in the endoscope housing are part of the endoscope. The endoscope allows, therefore, a spectral analysis of the captured light.

A method for tissue determination on the basis of a spectrometric measurement is disclosed by EP 2 725 967 Bi.

In addition, the expert article “3D-printed miniature spectrometer for the visible range with a 100×100 μm² footprint”, Andrea Toulouse, Johannes Drozella, Simon Thiele, Harald Giessen und Alois Herkommer, https://doi.org/10.37188/lam.2021.002 discloses a miniaturized spectroscopic measurement device.

Similarly, the expert article “Integrated spectroscopic analysis system with low vertical heightfor measuring liquid or solid assays” Yuhang Wan, Saoud A. Al-Mulla, Wang Peng, Kenneth D. Long, Benjamin A. Kesler, Patrick Su, John M. Dallesasse und Brian T. Cunnigham, http://www.elsevier.com/open-access/userlicense/1.0/discloses a spectroscopic measurement device.

Another construction type of a spectroscopic measurement device is known from EP 2 284 509 A1.

A multifocal lens arrangement that is directly applied onto an image sensor is known from WO 2018/072806 A1. The sensor is suitable for capturing images of an object while concurrently using different focal lengths.

EP 0 939 894 Bi discloses a miniature spectrometer having an electrical carrier plate on which a light source as well as a light capturing element are arranged. This arrangement is embedded into a transparent mass on which two lenses of different type are formed.

WO 2010/129324 discloses an endoscope having a working channel and a transparent end cap arranged on the distal end. Through the end cap an operation field can be illuminated by means of an LED light source. In addition, the end cap of the endoscope comprises multiple optical components, such as an optical fiber or an image sensor in addition to an associated lens.

US 2005/0154277 A1 also discloses a system having multiple lenses to which, however, multiple image sensors are assigned. This optical device can be part of a capsule that can be swallowed by a patient for examination of the gastrointestinal tract.

US 2009/0147076 A1 discloses an endoscope having an optical device for capturing images in multiple directions arranged on a distal end and a rectangular image sensor on the proximal end of the endoscope. The images coming from the different lenses are projected on different zones of the image sensor.

US 2010/0141380 A1 discloses an authentication method and a fingerprint sensor adapted for this purpose. It comprises an optical window having a planar top side for placing a finger thereon and a backside having a sawtooth profile. The elements of the sawtooth profile form a series of prisms. A light source serves for illumination of the back side. In proximity to the light source a first lens for creating an image on a CCD sensor as well as a second lens having a diffraction grating are provided that images a spectrum of the light reflected by the finger. The sensor comprises a first zone on which the image of the finger is projected as well as a second zone on which the spectrum is projected.

### SUMMARY

It is one object of the invention to provide an endoscope usable for diagnostics.

This object is solved by means of the endoscope for medical examination and/or treatment of a human or animal body as described herein.

The endoscope according to the invention comprises on its distal end a light capturing device and an illumination device. The light capturing device comprises at least one imaging first optical device configured for capturing a video stream or also individual photographs. An image sensor, particularly in form of a rectangular camera chip, is part of the light capturing device. Particularly suitable is an image sensor in MOS or CMOS technology.

The first optical device comprises an objective consisting of at least one optical element, for example a lens, that is configured to project a camera image onto a first zone of the image sensor. Thus, a first zone of the image sensor is assigned to the first optical device. The zone can have an angular or particularly also a round form, for example a circular form. The first zone is an image capturing zone. The image sensor can be particularly a rectangular image sensor. Its aspect ratio can be, for example, 16:9. The first zone can also be rectangular, however therefore have a different aspect ratio, for example 4:3 or 1:1 or another. Also, the first zone can have a form different from a rectangular form and can be, for example, polygonal or round.

Additionally, the light capturing device comprises a second optical device to which a second zone of the image sensor is assigned. This second zone can serve measurement purposes, whereby it forms a measurement zone. The measurement zone and the first zone (imaging zone) are arranged adjacent to one another on the image sensor. They can have different forms. While the imaging zone can be round, for example, the measurement zone can be configured in angular manner and can particularly use edge or corner regions of the image sensor.

The endoscope according to the invention thus comprises on its distal end a first optical device serving as image capturing device as well as a second optical device serving as measurement device. Both optical devices comprise preferably different light entrance windows and optical means arranged there, such as objectives in form of lenses, light passage gaps, optical gratings, light filters and the like. An image sensor is commonly assigned to both optical devices, wherein different zones of the image sensor are assigned to the first and second optical device.

Preferably, both optical devices are configured in a manner defining a respective optical axis, wherein these two optical axes are additionally preferably orientated parallel to one another. Alternatively, they can be orientated in a converging orientation in a distal direction away from the distal end of the endoscope. Due to these measures, it is possible to align the imaging optical device and the measuring optical device onto the same tissue area. In this manner, a measurement of tissue characteristics can be carried out concurrently with monitoring the tissue that defines an operation area, for example.

The second optical device serving measurement purposes can comprise two light entrance windows that are distant from each other, to which different second sub-zones of the image sensor are assigned. Preferably, the two light entrance windows of the second optical device are arranged on one side of the first optical device, while the light exit window of the assigned illumination device is arranged on the other side of the first optical device.

The light capturing device can have a third optical device, to which at least one third zone on the image sensor is assigned. The third optical device can be configured for carrying out a measurement task that is different from the measurement task of the second optical device.

The third optical device can comprise an individual light entrance window having a suitable objective or other optical means and can be configured for a desired measurement purpose. For example, the third optical device can be a device for carrying out tissue examinations.

The second and/or third optical device can be configured for carrying out the diffuse reflection spectroscopy. For carrying out the measurement, the instrument is brought into tissue contact with its distal end and particularly its light exit window. Thereby sub-zones of the image sensor having different distances from the light source can measure tissue arranged at different depths. The light source can be configured in narrow band or broad band illuminating manner. The emitted light can be entirely or partly in the range of visible light or entirely or partly in the range of infrared light. Information from and about deeper positioned tissue layers can be gained.

The second and/or third optical device can be alternatively configured for carrying out scattered light measurements. Thereby tissue sections having different distances from a light source are illuminated with different brightnesses by the probe positioned with distance to the tissue, so that a differential measurement of the light scattered by the tissue becomes possible. Thereby light is emitted with distance to the tissue and depending on how massively the surface scatters, different intensities can be measured at different distances from the light source.

The third optical device serving for measurement purposes can have two light entrance windows distanced from one another for this purpose, to which different third sub-zones of the image sensor are assigned. Preferably the two light entrance windows of the third optical device are arranged on two different sides of the first optical device. However, the light exit window of the assigned illumination device is arranged only on one side of the first optical device. The two different third zones of the image sensor are arranged at a distance to one another. For example, they can be arranged adjacent to opposite edges of the rectangular image sensor respectively. In addition, the two light entrance windows of the third optical device can be arranged at different distances from the light exit window of the illumination device. This allows to carry out differential measurement of tissue characteristics.

Another optical device, to which another zone of the image sensor can be assigned, can be reserved for another measurement method. Particularly this additional optical device can be configured for carrying out microscopy or microscopic measurement methods. Alternatives are possible. For example, the device can be configured for distance measurement or also for other measurement tasks.

The illumination device comprises at least one light source having a light exit direction that is preferably substantially parallel to the optical axis of the first (imaging) optical device. The light source can serve for image capturing as well as for carrying out tissue measurements. It can be provided that the light source has a larger emission angle than the capturing optics.

The light source can have a bandwidth that is at least as large as the bandwidth of the image sensor in relation to its light sensitivity. However, it is also possible to provide one or more light sources that emit only light having a bandwidth that is less than the sensitivity bandwidth of the image sensor and thus only serves a section thereof. These light sources can be configured in a manner to be switched on and off depending on the measurement task in order to temporarily concurrently or subsequently fulfill different measurement tasks. The light source can be particularly configured in a manner to emit visible light as well as infrared light. This spectral range can be provided by one single light source or can be distributed to multiple light sources.

The imaging zone is preferably arranged between two measurement zones that are assigned, for example, to the second optical device. The second optical device can be configured as spectrometer. Similarly, if needed, a third optical device can be provided that is a spectrometer or also a differential optical measurement device. Due to different spatial arrangement or also different configuration with regard to the spectral sensitivities of the different optical devices, different measurement tasks can be solved with their use. For example, the third optical device can be configured for carrying out the diffuse reflection spectroscopy. For this purpose of spectral filtering, the third optical device can comprise multiple filters arranged next to one another or a filter with position-dependent color, which filters the received light spectrally, for example in the wavelength range from 600 nm to 900 nm. The filters used for this purpose can be directly applied on the image sensor. Alternatively, one or more diffractive optical elements, such as a prism or grating, can be arranged with distance to the sensor.

In addition, the illumination device can have elements for structured illumination of the tissue, for example in form of a light grating or another pattern. For this purpose, the illumination device can comprise a laser diode, particularly an infrared laser diode, to which a diffractive optical element is assigned in order to project a light pattern onto the tissue. If this happens in the infrared range, the light pattern is not visible for the user. However, in this manner, a 3D model of the tissue surface can be created.

It is possible to assign two different optical devices to one and the same zone of the image sensor. For example, the imaging zone (first zone) of the sensor can be used concurrently for imaging by means of visible light, as well as for 3D model formation by means of infrared structured light. Also, other areas of the image sensor can be used in multiple manner.

The image sensor is preferably an RGB-IR-sensor. Such a sensor comprises light-sensitive elements (pixel) distributed over its rectangular light-sensitive surface and arranged in a grid. Preferably, thereby, light-sensitive elements for blue, green, red and infrared light are alternatingly distributed in suitable manner over the surface of the sensor. For this purpose a color filter matrix (Bayer matrix) for color display can be applied on the actual image sensor already by the manufacturer. The arrangement of the individual light-sensitive elements is preferably identical in all zones of the image sensor, that is in the imaging zone as well as the measurement zone. The color filter matrix can extend over the first zone serving for imaging as well as over the second or third zone serving for measurement purposes. Alternatively, the color filter matrix can also be omitted in the second and/or third zone or may also have been removed subsequently if provided by the manufacturer.

The image sensor can comprise pixels that are sensitive for two, three, four or also more different light wavelengths (that means colors) and thus can analyze light with a respective spectral resolution. In an image sensor having pixels, which are sensitive for four different colors, therefore, four different spectral lines (that means light components) of the received light can be distinguished.

It is in addition possible to detect a number of spectral lines with the same image sensor, which is remarkably higher than the number of colors that can be distinguished by the image sensor itself. This can be particularly carried out with multi-band-pass filters that allow light having different colors (that means light in multiple different wavelength ranges) to pass and apart therefrom absorb or reflect it. Preferably two or more multi-band-pass filters can be used that distinguish from each other with regard to their colors and which are arranged covering a larger group of pixels directly adjacent to each other or with distance next to each other in the light path, for example directly on the image sensor.

The differently colored components of the light that has passed the multi-band-pass filter equally impinge on the pixels of different color of the image sensor. The pixels are sensitive (sensitivity ranges) for different, for example four, wavelength ranges (color ranges). The sensitivity ranges can overlap. For this reason it can occur that passed light of a single color activates two pixel types of the image sensor that are adjacent to each other with regard to their wavelengths. It can also happen that in one sensitivity range of an image sensor type, two or more passage ranges of the multi-band-pass filters are provided. If multiple passage ranges of the multi-band-pass filter are present in one and the same sensitivity range, the light received by the same type of pixel can be assigned particularly easily to different spectral lines by means of calculation if in the sensitivity range only one and in the adjacent overlapping sensitivity range no passage range of the same multi-band-pass filter is present.

### BRIEF DESCRIPTION OF THE DRAWINGS

Additional details of advantageous embodiments and details of the invention are derived from the drawing, the description and the claims. The drawings show:

FIG. 1 an endoscope according to the invention in schematic illustration,

FIG. 2 a front view of the endoscope according to FIG. 1 in schematic illustration,

FIG. 3 the image sensor and the illumination device of the endoscope according to FIGS. 1 and 2 in schematic top view,

FIG. 4 a pixel arrangement of the image sensor of the endoscope,

FIG. 5 the optical conditions during carrying out a tissue examination with an endoscope tip placed onto the tissue and

FIG. 6 the endoscope during imaging use in a position distant from the tissue,

FIG. 7 the image sensor of the endoscope according to FIGS. 1 and 3 having an image zone and filters for configuration of a multi-channel spectrometer,

FIG. 8 the image sensor of FIG. 7 without filters,

FIG. 9 a first diagram for illustration of passage ranges of a first filter and sensitivity ranges of an image sensor,

FIG. 10 a second diagram for illustration of a second passage ranges of a filter and sensitivity ranges of the image sensor,

FIG. 11 a diagram for illustration of spectral lines that can be detected by means of the image sensor.

### DETAILED DESCRIPTION

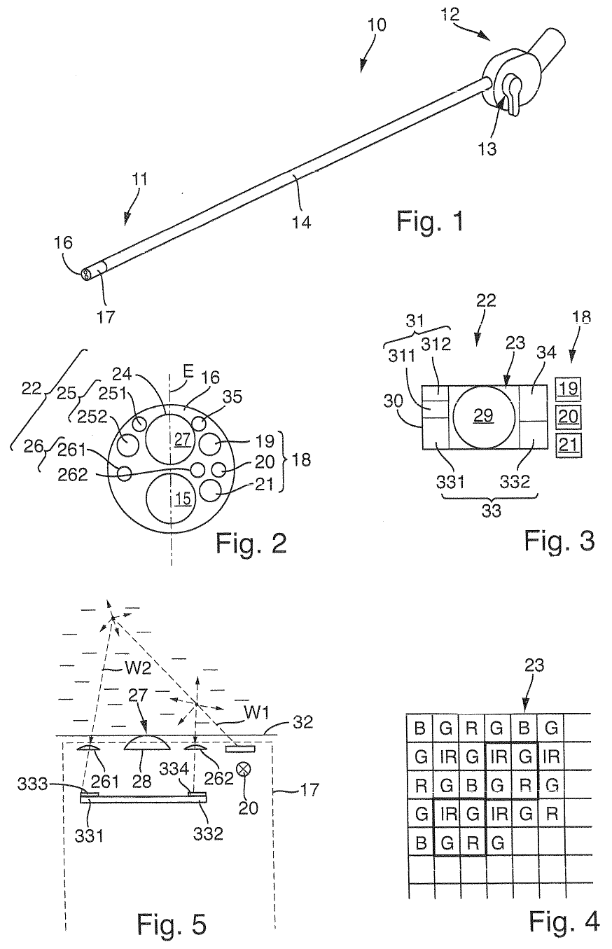

In FIG. 1 an endoscope 10 according to the invention is depicted in overview illustration. The endoscope 10 comprises a distal end 11, which is to be inserted into a patient, for example into a body lumen of the patient during carrying out an operation. The endoscope is then controlled from its proximal end 12 on which one or more operating elements 13 can be provided. Depending on the configuration of the endoscope 10, such operating elements 13 can be used for movement, particularly bending of a section of the distal end 11, for example. The longitudinal shank 14 of the endoscope can be apart therefrom rigid or as necessary also flexible.

The endoscope can have one or more working channels 15 which extend up to a face 16 of shank 14, wherein the face 16 is configured in planar or domed manner. The working channel 15 or the working channels 15 serve to locate not further illustrated probes or instruments therein that serve as tools for influencing biological tissue. Such instruments can be, for example, high frequency surgical instruments, cryosurgical instruments or other tools suitable for influencing tissue.

The endoscope 10 according to the invention comprises an extensive optical device 22 for the explanation of which reference is made to FIGS. 2 to 6. This optical device 22 is located in the distal end section 17 of the distal end 11, which itself is stable in form and thereby substantially cylindrically configured independent from whether shank 14 is stiff or flexible. The face 16 can, however, be domed and thereby be configured in semi-spherical manner, for example. Other shapes, particularly a substantially planar configuration having a rounded transition section to the cylindrical circumferential surface, are also possible and can be convenient for specific measurement tasks explained in the following.

Part of the optical device arranged in the end section 17 is at first an illumination device 18 comprising at least one, potentially however multiple, for example two or three, light sources 19, 20, 21 illustrated in FIG. 2 by means of their light exit windows. The light sources 19, 20, 21 can be light sources with identical optical characteristics, however, particularly also light sources with different characteristics. The differences can relate to:
the bandwidth and/or
the spectral composition of the emitted light,
the spatial distribution of the light, for example with regard to
   the opening angles or
   the created light patterns or also
temporal characteristics, such as
continuous light,
intermittent light as well as
points in time of the activation of the respective light sources.

For example, the first light source 19 can be a light source for the red to infrared wavelength range, for example, from 600 nm to 900 nm. The second light source 20 can be, for example, a white light source for the wavelength range of visible light or also a light source of colored light within the visible range. In addition, the second light source can be a light source comprising at least partly the range of visible light as well as at least partly the infrared range. In addition, the second light source can be a purely infrared light source. The light source 21 can be a white light source. It can be configured to uniformly illuminate the operation area. Alternatively, it can be configured to only illuminate a sub-area. Particularly, it can be configured as projection device and can be configured to create an image or light pattern in order to project, for example, a line image, a check image or another stationary or moving image onto the tissue located in front of the distal end 11 of the endoscope.

The light sources 19, 20, 21 can have coinciding light exit directions (optical axes) or also different light exit directions. In a preferred embodiment, at least one of the three light sources 19, 20, 21 comprises a light exit direction coinciding with the longitudinal direction of shank 14. Preferably, the light exit directions of two light sources or also all three light sources are orientated parallel to one another. The opening angles of the light cones exiting the light sources 19, 20, 21 can be of similar or also different dimension. Thereby, one or more light sources 19, 20, 21 can have a circular cone-shaped light exit with circular light exit surface respectively. One or more of the light sources 19, 20, 21 can also define light exit surfaces deviating therefrom, for example oval, semicircular or rectangular light exit surfaces.

The illumination device 18 is part of a sensor device suitable for image capturing and for carrying out measurements on the tissue, which in addition to the illumination device 18 comprises a light capturing device 22. The latter comprises an image sensor 23 individually illustrated in FIG. 3. The image sensor 23 can be a so-called camera chip having a plurality of light-sensitive cells (so-called pixels) arranged in a predefined raster. The individual pixels are typically sensitive for different light colors respectively, for example for blue, green and red. In a preferred embodiment, in addition, infrared-sensitive pixels are present. Accordingly, the pixels in FIG. 4 are characterized with “B” for blue, “G” for green, “R” for red and “IR” for infrared light. It is an RGB-IR-sensor.

Multiple optical devices 24, 25, 26 are assigned to the image sensor 23, which are illustrated in FIG. 2 based on their light entrance windows arranged on face 16.

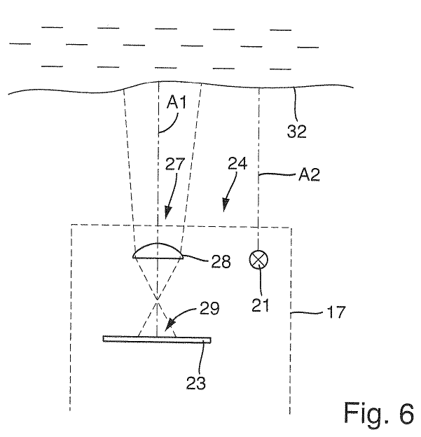

The first optical device 24 is an imaging device. It comprises an objective 27 illustrated by means of a lens 28 in FIGS. 5 and 6. The objective 27 can, however, comprise other and/or additional optical elements. The objective 27 is an imaging objective and configured to project an object image onto a first zone 29 of image sensor 23. This zone 29 can have a form deviating from the circumference of image sensor 23, as illustrated in FIG. 3, and can be particularly round, square or rectangular, for example with an aspect ratio of 4:3. Preferably, the zone 29 is arranged approximately centrally on the image sensor 23.

Between a narrow side 30 of image sensor 23 and zone 29, a second zone 31 can be provided serving as measurement zone. This second zone 31 can be divided into multiple sub-zones, for example two sub-zones 311 and 312, arranged between the first zone 29 and the narrow side 30. While the first zone 29 is an imaging zone, the second zone 31 is a measurement zone.

At least one of the light sources 19, 20, 21 is assigned to the first optical device 24. For example, light source 21 can serve to emit light for illumination of a tissue surface 32 (FIG. 6), wherein the first optical device 24 then serves to capture a respective image. However, the same or another light source can be assigned to the second optical device 25, for example the light source 19. While the light source 19 (or 20 or 21) are arranged on one side of a center line E, passing through the first optical device 24 and the working channel 15, the optical device 25 is preferably arranged on the other side of this line E. The device 25 can comprise one or two light entrance windows 251, 252 arranged next to one another that receive light and guide it onto the sub-zones 311, 312 of image sensor 23.

Another possibility of arrangement of a light source, for example the light source 20 and the light entrance windows 261, 262 of the third optical device, is depicted in FIGS. 2 and 3. Assigned to the light entrance windows 261, 262 of third optical device 26 is a third zone 33 of image sensor 23, which in turn can comprise two sub-zones 331, 332. Preferably, the two sub-zones 331, 332 are arranged on both sides of imaging zone 29. Thus, measurement zones or sections 331, 332 thereof are provided on both sides of imaging zone 29.

In addition, in a remaining section of image sensor 23, a fourth measurement zone 34 can be provided to which an individual objective 35 is assigned as well, which can serve for microscopy, for example.

On the rectangular image sensor 23, therefore, at least one zone 29 serving for imaging as well as at least one additional zone is provided, preferably multiple additional zones 31, 33, 34 are provided, serving for measurement of tissue characteristics. While the imaging zone 29 is preferably symmetrically arranged, that means centered on the image sensor 23, the zones 31, 33, 34 of image sensor 23 suitable for carrying out measurement tasks can be arranged symmetrically or asymmetrically on both sides of the imaging zone 29.

The image sensor 23 is preferably a semiconductor sensor having a plurality of pixels which has the same uniform configuration for the imaging zone 29 as well as the measurement zones 31, 33, 34.

The endoscope 10 described so far can be used variously:

In FIG. 6 the endoscope 10 or its end section 17 is schematically depicted during use for imaging. It is positioned with distance to a surface section 32. The surface section 32 is illuminated by means of the third light source 21. The tissue image is projected on the image sensor 23 and there particularly on the imaging zone 29 via objective 27. The image sensor 23 therefore creates images or a video stream, that can be transmitted by means of not further illustrated means to the proximal end of endoscope 12 and from there toward an image display device, for example VR glasses, a screen or the like.

If the light source 21 is a light source for structured illumination or if it can be switched to emit structured light, it can be used to project a light pattern on the tissue surface 32. The resulting image can be supplied to an image processing device that calculates a 3D model of the tissue surface 32 therefrom and provides it for rendering.

It is possible to configure light source 21 so that it emits non-structured light in the visible light range and structured light in the infrared (non-visible) light range. In this manner, the image sensor 23 can on one hand create an optical image for representation for a surgeon and on the other hand create an infrared image from which an image processing device can determine a relief image, that means a 3D model of the tissue surface 32, and provide it for further processing or observation.

FIG. 5 illustrates an operating mode in which no imaging, but a tissue measurement is carried out. For this purpose, the distal face 16 of endoscope 10 is placed onto the tissue surface 32. The operation is explained by way of example of light source 20 that cooperates with the two parts 261, 262 of optical device 26. For example, light source 20 emits broadband infrared light that enters the tissue and is scattered there. The light reaches the light entrance windows of the two parts 261, 262 on paths W1, W2 of different lengths. Owing to the longer path W2, the sub-zone 331 receives a light signal that has been scattered in deeper tissue layers far from the surface. Thereby, by means of comparison of the two signals, the scattering of the tissue, the type of the illuminated tissue layers and in this manner also the proximity to an organ can be determined. This applies particularly if the organ located in the tissue has light scattering characteristics different from the embedding surrounding tissue. In addition, light is absorbed at different degrees due to different light wavelengths. By means of the spatial differential measurement, it is in addition possible to distinguish scattering and absorption if the present tissue type comprises a homogeneous structure.

For carrying out this differential diffuse reflection spectroscopy, alternatively, light sources can be provided for preferably not too narrow band infrared light or for visible colored or white light or for light which comprises portions in the visible as well as in the infrared spectral range.

At least one of the zones 31, 33, or at least one sub-zone 311, 312, 331, 332 thereof, serving for measurement purposes, can be configured for carrying out spectrometric measurement tasks. For this purpose, in the individual zones 31 and/or 32 serving for measurement, spectrally filtering elements can be applied or arranged in relation thereto. In the simplest case narrow band color filters 333, 334 (FIG. 5), for example interference optical filters, are directly applied on the image sensor 23, wherein respective individual pixels or pixel groups are provided with one filter for a selected wavelength. In this manner, a spectral analysis of the light scattered back from the tissue surface 32 can be carried out concurrently to imaging. Instead of individual filters arranged next to each other, also a so-called linearly variable filter can be provided, the pass wavelength of which varies along a line, preferably a straight line.

With the endoscope 10, according to the invention, different measurement tasks can be concurrently or subsequently carried out. If the endoscope 10 is positioned with its face distant to the tissue surface 23, the following can be carried out:
imaging by means of the first optical device 24,
2D-imaging with visible light and concurrent 3D-detection by means of structured illumination with IR light,
imaging by means of optical device 24 and differential diffuse reflection spectroscopy with third optical device 26,
imaging by means of optical device 24 and microscopy by means of fourth optical device 35,
imaging by means of optical device 24, diffuse reflection spectroscopy by means of third optical device 26 and microscopy by means of fourth optical device 35.

However, if the endoscope 10 is placed with its face on the tissue surface 23, the following can be carried out:
diffuse reflection spectroscopy by means of the second optical device 25 and/or
diffuse reflection spectroscopy by means of the third optical device 26.

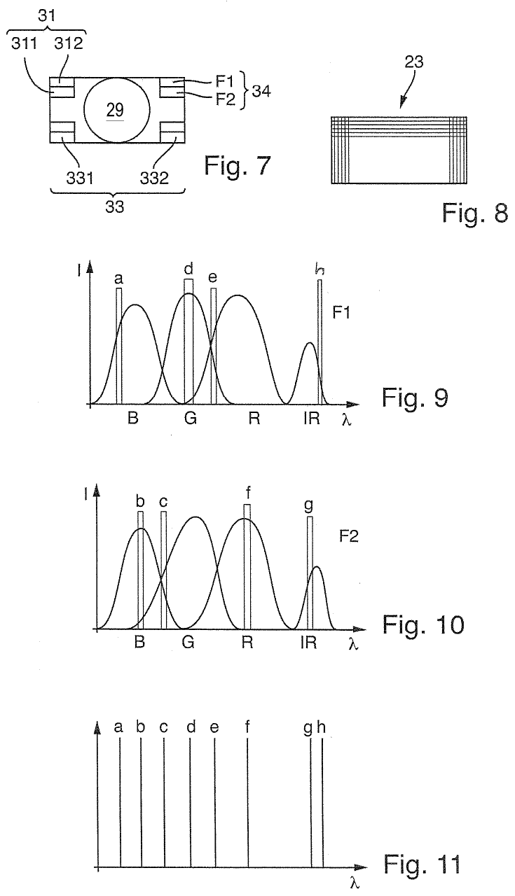

As mentioned, zones of the image sensor 23 that are not used for imaging, different to the imaging zone 29, can be used for measurement purposes. Thereby it is possible to carry out spectroscopic measurements by means of the image sensor 23. A specifically advantageous possibility for this is disclosed in FIGS. 7 to 11 in which at least one of the measurement zones 31, 33, 34 is used as spectrometer and is thereby particularly configured as a spectrometer appropriate for analysis of diffuse light.

The image sensor 23 comprises multiple pixels B (blue), G (green), R (red), infrared (IR) that are sensitive for different wavelength ranges. They can be arranged according to the scheme of FIG. 4 or in another scheme. In addition, image sensor 23 can also comprise a different number of pixel types, for example only two or three. The sensitivity ranges of the individual pixels B, G, R, IR are illustrated in FIGS. 9 and 10. As apparent, all sensitivity ranges have respectively one maximum, whereby the light sensitivity of the respective pixels B, G, R, IR decreases toward both sides of the maximum. The sensitivity ranges of adjacent pixels B/G, G/R can overlap remarkably as illustrated in FIGS. 9 and 10.

Two filters F1, F2, which are not arranged in overlapping manner, are part of the spectrometric measurement zone 34. They can be arranged on the image sensor 23 directly adjoining one another or also in a certain distance to each other. The filter F1 is a first multi-band-pass filter that allows light to pass within passage bands a, d, e and h, illustrated in an idealized manner in FIG. 9, but absorbs or reflects light of other wavelengths. In FIG. 10 the passage characteristic of the second multi-band-pass filter F2 is illustrated that, as illustrated in an idealized manner there, allows light within passage bands b, c, f and g to pass, while other wavelength ranges are blocked or absorbed or reflected.

Now, for light evaluation two pixel groups exist, namely the group of pixels B, G, R, IR that are illuminated by the light of multi-band-pass filter F1 and a second group having pixels B, G, R, IR that are illuminated by the light of multi-band-pass filter F2. As apparent from FIG. 9, thereby light, for example in the passage band e, can induce a signal in the pixels G as well as in the pixels R of the group of filter F1. Similarly, light of the passage band c can induce signals in the pixels B, G of the group below filter F2. Although light of both passage bands d and e activate pixels G of green sensitivity and although light from the passage bands b, c activates the pixels B of blue sensitivity, the spectral components of the passage bands d, e as well as the spectral components of passage bands b, c can be separated from one another. This is demonstrated in the following by way of example of the intensities I of the spectral components of the passage bands c and e present respectively in the overlapping range: Specifically simple is the separation of the spectral components of the passage bands d and e of filter F1, because in addition to the passage band e inside the overlapping range only one single passage band d is located adjacently. Further, because in one of the adjacent sensitivity ranges of the pixels G and R, no passage band of the same filter F1 and in the other of the adjacent sensitivity ranges of the pixels G and R, no passage band exists. The passage band F is assigned to the second filter F2 and can there be read on the red pixel R in unbiased manner.

Similarly, light of passage bands b and c below filter F2 activates both pixels B and G. However, only for the pixel B an additional passage band b is present. The green pixels G receive, however, only light from the passage band c while light from the passage band d is gained via the pixels of the first filter area F1.

The intensity I of the passage bands c and e can be determined as follows:

The relation of the signals of pixels B and G in the overlapping range at the wavelength of the passage band c is known from data sheets and is here denoted as F_{between GB at c}. The signal Gat c supplied from the green pixel G for the passage band c is multiplied by factor F_{between GB at c} in order to obtain the intensity of Bat c. The signals or intensities I_{c} and I_{b} can be calculated as follows:

$${B}_{at c} = G \cdot {F}_{between GB at c} \\ {I}_{c} = {B}_{at c} + G \\ {I}_{b} = B - {B}_{at c} \\ {G}_{at e} = R \cdot {F}_{between RG at e} \\ e = {G}_{at e} + R \\ d = G - {G}_{at e}$$

The spectrum having eight spectral lines illustrated in FIG. 11 is obtained thereby. On additional surface areas of the image sensor 23 additional multi-band-pass filters can be provided in order to further improve the spectral resolution. The condition for this is that the ratios of the spectral sensitivities of pixels, which are adjacent with regard to their wavelengths, are known in the overlapping range and only one multi-band-pass double occupancy exists per channel (RGB). These double occupancies are d and e in FIG. 9 and b and c in FIG. 10. It is helpful if in the same or at least in one of the respective other filters in the adjacent color channel (here R and G) no channel occupancy (no passage range of the respective filter) exists.

The invention relates to an endoscope 10 that is configured for imaging as well as for tissue analysis. For this purpose, it comprises an image sensor 32 to which multiple optical devices 24, 25, 26 are arranged in order to carry out different tasks with one and the same image sensor 23, particularly the task of imaging and measurement tasks concurrently or temporally graded.

### LIST OF REFERENCE SIGNS

10 endoscope
11 distal end of endoscope 10
12 proximal end of endoscope 10
13 operating element of endoscope 10
14 shank of endoscope 10
15 working channel of endoscope 10
16 face of shank 14
17 end section
18 illumination device
19 first light source
20 second light source
21 third light source
22 light capturing device
23 image sensor
R red sensitive pixel
G green sensitive pixel
B blue sensitive pixel
IR infrared sensitive pixel
24 first optical device
25 second optical device
251, 252 parts of the second optical device
26 third optical device
261, 262 parts of the third optical device
27 objective of optical device 24
28 lens of objective 27
29 first zone (imaging zone)
30 narrow side
31 second zone (measurement zone)
311, 312 sub-zone of second zone 31
32 tissue surfaces
33 third zone (measurement zone)
331, 332 sub-zone of third zone 31
333, 334 color filter
F1 first multi-band-pass filter
F2 second multi-band-pass filter
a-h passage bands of filters F1, F2
34 fourth zone (measurement zone)
35 objective

1. An endoscope (10), comprising:
a longitudinal shank (14) comprising an illumination device (18) and a light capturing device (22) at a distal end (11) of the longitudinal shank;
wherein the light capturing device (22) comprises a first imaging optical device (24) and a second optical device (25) to which a first zone (29) and a second zone (31) of a common image sensor (23) are respectively assigned, wherein the first zone (29) defines an imaging zone (29) and the second zone (31) defines at least one measurement zone (31) that is different from the imaging zone on the image sensor (23).

2. The endoscope according to claim 1, wherein the first imaging optical device (24) and the second optical device (25) are configured to individually define one optical axis, respectively.

3. The endoscope according to claim 2, wherein the optical axes are parallel to one another.

4. The endoscope according to claim 1, wherein the light capturing device (22) comprises a third optical device (26) to which at least one third zone (33) on the image sensor (23) is assigned that forms another measurement zone different from the imaging zone (29).

5. The endoscope according to claim 1, wherein the second optical device (25) comprises two light entrance windows (251, 252) to which different sub-zones (311, 312) of the second zone (31) of the image sensor (23) are assigned.

6. The endoscope according to claim 5, wherein the two light entrance windows (251, 252) are positioned at different distances from the illumination device (18).

7. The endoscope according to claim 1, wherein the illumination device (18) comprises at least one light source (19, 20, 21) having a light exit direction (A2) that is defined to be parallel to an optical axis (A1) of the first optical device (24), wherein the at least one light source (21) is arranged with a lateral distance from the first optical device (24).

8. The endoscope according to claim 1, wherein the imaging zone (29) is arranged between two measurement zones (31/34; 331, 332).

9. The endoscope according to claim 1, wherein the second optical device (25) is configured for carrying out a spectrometric measurement and/or for carrying out a differential optical measurement.

10. The endoscope according to claim 1, wherein the second optical device (25) is arranged relative to the first optical device (24) using sub-areas (251, 252) arranged asymmetrically on the image sensor (23) and/or wherein a third optical device (26) is arranged relative to the first optical device (24) using sub-areas (261, 262) arranged symmetrically on the image sensor (23).

11. The endoscope according to claim 9, wherein at least two color filters (333, 334, F1, F2) matching with different wavelengths are applied on the image sensor (23) to form a spectrometer.

12. The endoscope according to claim 11, wherein the image sensor (23) comprises multiple pixels (R, G, B, IR) sensitive to different wavelength ranges and arranged in a periodic pattern and wherein the at least two color filters (F1, F2) are multi-band-pass filters having multiple passage bands (a, d, e, h; b, c, f, g), respectively.

13. The endoscope according to claim 12, wherein at least two of the different wavelength ranges of the multiple pixels (R, G, B, IR) overlap each other, so that light having a wavelength in an overlapping range produces a signal in both pixels that are sensitive in the overlapping range.

14. The endoscope according to claim 12, wherein at least one of the multi-band-pass filters (F1, F2) comprises at least one passage band (e, c) that is provided between adjacent wavelength ranges of respective pixels (R, G, B, IR) of the multiple pixels.

15. The endoscope according to claim 11, wherein the at least two color filters (F1, F2) are configured as multi-band-pass filters that are each arranged in a manner covering respectively one group of multiple pixels (R, G, B, IR) that are sensitive to different wavelength ranges, wherein each group of multiple pixels comprises multiple pixels (R, G, B, IR) of each wavelength range, respectively.
